(19) United States
(12) Patent Application Publication
(10) Pub. No.: US 2006/0258708 A1
(43) Pub. Date: Nov. 16, 2006

(54) Method for treating Parkinson's disease and other neurological diseases

(76) Inventor: Peter J. Andrulis JR., Bethesda, MD (US)

Correspondence Address: Isaac A. Angres, Suite 301, 2001 Jefferson Davis Highway, Arlington, VA, 22202, US

(21) Appl. No.: 11/433,531

(22) Filed: May 15, 2006

Related U.S. Application Data
(60) Provisional application No. 60/681,021, filed on May 16, 2005.

(51) Int. Cl. A61K 31/454; A61K 31/522; A61K 31/198; A61K 31/192; A61K 31/137

(52) U.S. Cl. 514/323; 514/570; 514/263.32; 514/649; 514/567

(57) ABSTRACT

A method for treating a central nervous system or peripheral nervous system dopaminergic deficit state or other neurological deficit state in a mammalian organism in need of such treatment, said method comprising administering to said mammal an amount of thalidomide effective in the treatment of a dopaminergic deficit state or other neurological deficit state and for a time sufficient to achieve a suitable blood level to treat said dopaminergic deficit state or other neurological deficit state.

This application claims the priority benefit under 35 U.S.C. section 119 of U.S. Provisional Patent Application No. 60/681,021 entitled “Method For Treating Parkinson's Disease And Other Neurological Disorders” filed May 16, 2005, which is in its entirety herein incorporated by reference.

### FIELD OF THE INVENTION

The present invention is directed to compositions, and methods for alleviating the symptoms associated with neurological disorders, i.e., Parkinson's disease and other neurological disorders found to be associated with inflammation. In one specific aspect, the present invention is directed to a method for treating Parkinson's disease with thalidomide.

In another specific embodiment of the invention, Parkinson's disease is treated with thalidomide alone or in conjunction with nonsteroidal anti-inflammatory agents, a dopamine agonist, and/or glial-derived neurotropic factor (GDNF). Combinations of thalidomide with other anti-Parkinson's agents such as levodopa and levodopa with carbidopa (Sinemet®, Atamet®) are part of the invention. Combinations of thalidomide with catechol-O-methyl transferase (COMTAN®) agonists such as pramipexole (Mirapex®), ropinirole (Requip®), among others; and/or a GDNF conjugate and other cytokine or growth factor conjugates that cross the blood-brain-barrier (BBB) are also part of the invention.

### BACKGROUND OF THE INVENTION

Thalidomide (N-phthalidoglutarimide) was first synthesized in 1953 by researchers at Chemie Grunenthal in Germany. It was marketed in Europe in 1956 as a sedative/hypnotic drug. Thalidomide is orally administered. It is poorly absorbed in humans. When 100 to 200 milligrams (mg) of thalidomide is ingested by humans, a maximal blood concentration of 0.9 to 1.5 mg/liter is attained 4 to 6 hours later. Thalidomide is extensively distributed throughout the body but does not selectively localize in any tissue. Thalidomide breaks down by spontaneous hydrolysis; however, the hydrolyric cleavage in serum is much slower than in vitro at pH 7.4. This may be due to thalidomide being highly bound to plasma proteins. Thalidomide metabolites are renally excreted. In a series of animal studies, racemic thalidomide has not been toxic, however, side effects in humans have included somnolence, teratogenicity and, upon extended administration, peripheral neuropathy. The teratogenic potential of thalidomide resulted in a variety of malformations of fetuses (phocomelia) and the subsequent removal of thalidomide from the market. The mechanism of the teratogenic effect is not known; however, it will occur when thalidomide is taken between the 35th and 50th day of the menstrual age of the embryo.

In addition to the sedative effect, thalidomide has exhibited an immunomodulatory effect which has resulted in its therapeutic testing or use against the following conditions: rheumatoid arthritis, acute and chronic graft versus host disease, aphthosis, cold hemagglutinin disease, colitis, cutaneous lupus erythematosus, erythema nodosum leprosum, erythema multiform, histiocytosis, immune complex vasculitis, Jessner-Kanof's disease, lichen planus, pemphigoid disorders, photodermatoses, prurigo nodularis, pyoderma gangrenosum, sarcoidosis and Weber Christian's disease. The exact immunomodulatory effect of thalidomide on a molecular level has not been determined; however, a number of observations in this regard have been made. Coulsen et al. (Clin. Exp. Immunol., 7:241, 1970) showed thalidomide derivatives could inhibit production of transformed cells in mixed lymphocyte culture, whereas the lymphocyte response to phytohemagglutinin was not inhibited. In another study by Moncada et al., (Int. J. Leprosy,:53:209, 1985) thalidomide was associated with increases in previously suppressed CD4 lymphocyte counts in erythema nodosum leprosum. Thalidomide has also been observed by Sampaio et al. (J. Exp. Med., 173:699, 1991) to inhibit tumor necrosis factor alpha production by stimulated monocytes. Elevated levels of tumor necrosis factor alpha in erythema nodosum leprosum were reduced by treatment with thalidomide (Sampaio et al., J. Exp. Med., 175:1729, 1992). Tumor necrosis factor alpha has been demonstrated to induce expression of HIV from cell lines (Poli et al., Proc. Nat. Acad. Sci., USA, 87:782, 1990). Blood tumor necrosis factor levels are high in HIV-infected individuals (Lahdevirta et al., Am. J. Med., 85:289, 1988). Thalidomide has been shown to reduce HIV production in peripheral blood mononuclear cells of HIV-infected patients. Thalidomide also inhibited HIV expression from infected cells lines induced with tumor necrosis factor alpha (Schauf et al., Intl. Sci. Conference on Antimicrobial Agents and Chemotherapy, Anaheim, Calif., 1992). (Reference: U.S. Pat. No. 5,425,179, July 1995.)

Parkinson's disease is the second most common neurological disease, following Alzheimer's. (Nussbaum et al., J Med N Eng., 348:1356-1364, 2003.) Onset of Parkinson's disease typically occurs between the ages of 55-60, affecting both men and women. Approximately 2.7 million people in the U.S., Japan, France, Germany, Italy, Spain and the UK suffer from Parkinson's disease. 85% of these are over 65 years old. Given the increased proportion of these elderly persons, the Parkinson's disease population in these countries is expected to grow by 2% per year in 2006. (H. Lundbeck A. S, Ottiliavej 9, DK-2500 Copenhagen Valby.) Parkinson's disease is a neurodengerative disease pathologically characterized by the progressive destruction of dopamine-producing nerve cells located in the substantia nigra of the brain and elsewhere and correlated with the presence of ubiquinated protein deposits in the cytoplasm of neurosn (Lewy bodies) (Kuzuhara et al., Acta Neuropathol (Berl) 75:345-353, 1988; Tanner et al., Neurol Clin 14:317-335, 1996.). Dopamingeric decline is observed clinically by parkinsonism (resting tremor, bradykinesia, rigidity, and postural instability). (Hoehn et al. Neurology, 17:427-442, 1967.) evidenced in movement disorders, among other factors. Parkinson's disease is divided into five stages by Hoehn and Yahr that range from mild, inconvenient movement disorders to slow and labored movement to complete invalidism requiring constant nursing care. This rating system has been replaced by the Unified Parkinson's Disease Rating Scale (UPDRS), a complex rating tool with numerical grades assigned throughout the longitudinal course of Parkinson's disease.

Although causality of the disease is not known, McGeer hypothesizes that chronic inflammation plays an important role in the pathogenesis of Parkinson's disease. (McGeer et al., Parkinsonian Relat Disord. 10(1):S3-7, 2004.) Significant increase of inflammatory cytokines such as TNF-alpha, I1-1beta and IFN-gamma in glial cells in the substantia nigra of Parkinson's patients is observed. (Hunot et al., J. Neurosci, 19(9):3440-7, 1999).) Changes in the levels of cytokines, neurotrophins, and apoptosis-related proteins in the nigrostriatal regions of Parkinson's disease may be involved in the degeneration of dopamine. (Nagtsu et al., J Neural Transm Suppl., 58:143-151, 2000). Markedly increased levels of cytokines are observed in the nigrostriatal dopamine degeneration regions and in the cerebrospinal fluid of Parkinson's patients suggesting neuroprotective therapy including nonsteroidal anti-inflammatory drugs (NSAIDS). (Nagtsu et al., J Neural Transm Suppl. 60:277-90, 2000.). Immunomodulating factors are inferred in the pathogenesis of sporadic PD as evidenced in a molecular genetic approach. (Kruger et al., J Neural Transm., 107(5):553-62, 2000.) Inhibition of inflammatory processes may represent the therapeutic target to reduced neuronal degeneration in Parkinson's disease. (Hirsch et al., Ann NY Acad Sci., 991:214-28, 2003.) The authors reported elevated levels of tumor necrosis factor in Parkinson's patients compared to controls and indicated elevated circulating tumor necrosis factor may be derived from the local central nervous system inflammatory reaction found in Parkinson's patients and may account for some of the systemic manifestations of Parkinson's disease such as severe forgetfulness, abnormality of gate and tremors.

Chronic inflammation is thought to play a causative role in other neurological diseases, such as, for example, Alzheimer's disease: β-deposits in the brain may trigger an inflammatory response resulting in the destruction of both damaged and healthy nerve tissue (Schnabel, Science, 260:1719, 1993). A number of investigators have observed that the brains of patients with Alzheimer's disease exhibit many of the classical markers of immune-mediated damage. These have included increased numbers of microglia (cells believed to be the functional equivalent to macrophages in the central nervous system) (Stryren et al., Exp. Neurel., 110:93, 1990) and astrocytes expressing inflammatory reactants interleukin 1 and alpha 1 antichymotrypsin (Abraham et al., Cell, 52:487, (1988). Complement proteins of the classical pathway have been immunohistochemically detected in Alzheimer's brain tissue and are most often associated with the .beta.eta plaques. Rogers et al., (Proc. Nat. Acad. Sci., USA, 89:10016, 1992) presented evidence that beta protein activates the classical complement pathway without mediation of immunoglobulin, thereby contributing to the development of the inflammatory process. In another study by Fillit et al., (Neurosci. Lett., 129:318, 1991) levels of tumor necrosis factor alpha were measured in both patients with Alzheimer's disease and age-matched controls by enzyme-linked immunosorbent assay and cytotoxicity bioassay.

### SUMMARY OF THE INVENTION

The primary object of the present invention is the treatment of neurological disorders by administering thalidomide to a patient in need of such treatment.

Another object of the present invention is to provide a therapeutic method for alleviating the symptoms of cognitive neurological decline by administering a therapeutically effective amount of thalidomide.

An additional object of the present invention is to treat Parkinson's disease with a therapeutically effective amount of thalidomide.

Another object of the present invention is to treat Parkinson's disease or other neurological disorders with thalidomide in combination with a glial-derived neuotropic factor (GDNF) conjugate that crosses the BBB in combination with other drugs.

A further object of the invention is to treat Parkinson's disease with a pharmaceutical composition comprising thalidomide, a dopamine agonist and a GDNF-conjugate or other cytokine or growth factor conjugates that cross the BBB.

Still another object of the invention is to treat Parkinson's disease with a pharmaceutical composition comprising thalidomide conjugated to a dopamine agonist and/or to a GDNF-conjugate or other cytokine or growth factor conjugates that cross the BBB.

Another object of the invention is to treat Parkinson's disease with pharmaceutical composition comprising thalidomide and a dopamine agonist and/or a GDNF-conjugate or other cytokine or growth factor conjugates that cross the BBB.

Still another object of the invention is to treat Parkinson's disease with a pharmaceutical composition comprising thalidomide and separate pharmaceutical compositions comprising a steroidal anti-inflammatory carboxylic acid.

The present inventor has now discovered a method for treating central nervous system or peripheral nervous system dopaminergic deficit states or other neurological deficit states in a mammal. The method comprises administering to a mammal an amount of thalidomide alone or in combination with compounds selected from the group consisting of non-steroidal anti-inflammatory carboxylic acids (NSAIDs), dopamine agonists, pentoxyphylline and a GDNF-conjugate or other cytokine or growth factor conjugates that cross the BBB effective in the treatment of dopaminergic deficit states or other neurological deficit states and for a time sufficient to achieve a suitable blood level to treat said dopaminergic deficit state(s) or other neurological deficit states.

The present inventor has also discovered a pharmaceutical composition of matter for treating said dopaminergic deficit states or other neurological deficit states in a mammalian organism in need of such treatment, said composition comprising a unit dosage amount of thalidomide alone or in combination with the above mentioned compounds and pharmaceutically acceptable carrier.

### DESCRIPTION OF THE PREFERRED EMBODIMENTS

The present invention deals with a method for treating a central nervous system or peripheral nervous system dopaminergic deficit state or other neurological deficit states in a mammalian organism in need of such treatment, said method comprising administering to said mammal an amount of thalidomide effective in the treatment of a dopaminergic deficit state or other neurological deficit states and for a time sufficient to achieve a suitable blood level to treat said dopaminergic deficit state or other neurological deficit states.

Additionally, the present invention relates to a method of treating the symptoms of neurological decline in a mammal which comprises administering to a mammal affected with said neurological decline a therapeutically effective amount of thalidomide.

Furthermore, the present invention provides a method of treating a mammal suffering from neurological decline with thalidomide and independently with other agents selected from the group consisting of levodopa, carbidopa, non-sterodial anti-inflammatory agents, sterodial anti-inflammatory agents, dopamine agonists, and/or pentoxyphylline and/or a GDNF-conjugate or other cytokine or growth factor conjugates that cross the BBB.

The invention also provides a method for treating the symptoms of neurological decline in a mammal which comprises administering to a mammal affected with said neurological decline a therapeutically effective amount of a mixture of thalidomide with a compound selected from the group consisting of levodopa, carbidopa, non-steroidal anti-inflammatory carboxylic acids (NSAIDs), inhibitors, dopamine agonists, pentoxyphylline and/or a GDNF-conjugate or other cytokine or growth factor conjugates that cross the BBB.

The present invention further provides a method for treating Parkinson's disease in a mammal, said method comprising administering to said mammal a therapeutically effective amount of thalidomide.

The therapeutically effective amounts of thalidomide are typically 50 mg to 200 mg and preferably 50 mg to 100 mg.

When thalidomide is used in combination with NSAIDs or steroidal anti-inflammatories (SAIDs), the amount of thalidomide is typically in the range of about 50 mg to 200 mg while the NSAIDs are present in the range of about 50 mg to 300 mg and the SAIDs are present in the range of 10 mg to 60 mg. For example, an effective combination for treating Parkinson's is a gelatin capsule containing 200 mg of thalidomide and 200 mg of ibuprofen given at bedtime daily.

The precise amount of thalidomide alone or with the other active materials mentioned above will vary depending, for example, on the condition for which the drug is administered and the size and kind of the mammal. Generally speaking, the thalidomide can be employed in any amount effective in the treatment of central nervous system or peripheral nervous system dopaminergic deficit states or other neurological deficit states. The symptoms of these states, including Parkinson's type are improved.

For humans, typical effective amounts of thalidomide for use in the unit dose compositions of the present invention range from about 50 mg to 200 mg per 24 hours; however greater amounts may be employed, if tolerable. This range is based on administration to a 70 Kg human. A preferred amount is 100 mg to 200 mg of thalidomide per 24 hours.

As mentioned above, thalidomide may be given alone or in combination with other drugs which are also useful in the treatment of central nervous system or peripheral nervous system dopaminergic deficit states or other neurological deficit states.

For example, when thalidomide is combined with NSAIDs or steroidal anti inflammatories, a typical formulation contains from about 100 mg to about 200 mg of thalidomide, and from about 100 mg to 300 mg of NSAIDs or 10 mg to 60 mg of SAID. The preferred non-steroidal anti-inflammatory is selected from the group consisting of arylpropionic acids, arylacetic acids, biphenylcarboxylic acids, diphenylether carboxylic acids, the salicylates, and the fenamic acids.

The non-steroidal carboxylic acids can be characterized into four groups as shown in U.S. Pat. No. 545,179, July 1995): (1) The propionic acid derivatives; (2) the acetic acid derivatives; (3) the fenamic acid derivatives; and (4) the biphenylcarboxylic acid derivatives or a pharmaceutically acceptable salt thereof.

The propionic acid derivatives which may be used comprise: ibuprofen, ibuprofen aluminum, indoprofen, ketoprofen, naproxen, benoxaprogen, flurbiprofen, fenoprofen, fenbufen, pirprogen, carprofen, oxaprozin, pranoprofen, miroprofen, tioxaprofen, suprofen, alminoprofen, tiaprofen, fluprofen, and bucloxic acid. Structurally related propionic acid derivatives having similar analgesic and anti-inflammatory properites are also intended to be included in this group. Thus, “propionic acid derivatives” as defined herein are non-narcotic analgesics/non-sterodial anti-inflammatory drugs having a free —CH(CH₃)COOH or —CH₂CH₂COOH group (which optionally can be in the form of pharmaceutically acceptable salt group, e.g., —CH(CH₃)COO⁻Na⁺ or —CH₂CH₂COO⁻Na⁺), typically attached directly or via a carbonyl function to a ring system, preferably to an aromatic ring system.

The acetic acid derivatives which may be used comprise: indomethacin, which is a preferred NSAID, sulindac, tolmetin, zomepirac, diclofenac, fenclofenac, alclofenac, ibufenac, isoxepac, furofenac, tiopinac, zidometacin, acematacin, fentiazac, clidanac, oxpinac, and fenflozic acid. Structurally related acetic acid derivatives having similar analgesic and anti-inflammatory properties are also intended to be encompassed by this group.

Thus, “acetic acid derivatives” as defined herein are non-narcotic analgesics/non sterodidal anti-inflammatory drugs having a free —CH₂ COOH group (which optionally can be in the form of a pharmaceutically acceptable salt group, e.g., —CH₂COO⁻Na⁺), typically attached directly to a ring system.

The fenamic acid derivatives which may be used comprise: mefanamic acid, meclofenamic acid, flufenamic acid, niflumic acid, and tolfenamic acid. Structurally related fenamic acid derivatives having similar analgesic and anti-inflammatory properties are also intended to be encompassed by this group.

Thus, “fenamic acid derivatives” as defined herein are non-narcotic analgesic/non-steroidal anti-inflammatory drugs which contain the basic structure as shown in U.S. patent No. to Andrulis, et al. which can bear a variety of substitutents and in which the free —COOH group can be in the form of a pharmaceutically acceptable salt group, e.g., —COO⁻Na⁺.

The biphenylcarboxylic acid derivatives which can be used comprise: diflunisal and flufenisal. Structurally related biphenylcarboxylic acid derivatives having similar analgesic and anti-inflammatory properties are also intended to be encompassed by this group.

Thus, “biphenylcarboxylic acid derivatives” as defined herein are non-narcotic analgesics/non-steroidal anti-inflammatory drugs which contain the basic structure as shown in US patent No. to Andrulis, et al. which can bear a variety of substituents and in which the free —COOH group can be in the form of a pharmaceutically acceptable salt group, e.g., —COO—Na⁺. Typical acids include ibuprofen, diflumisol, fenoprotenin acid, meclofenic acid, mefenamic acid, naproxen, suliudor, indomethacon, talmetin, fenbufen, ketoprofen, indoprofen, fluprofen, benozaynofen, pirprofen, miroprofen, thioxaprofen, aspirin, choline magnesium salicylate as well as those NSAIDs disclosed in the “Physicians Desk Reference” (1992 edition) whose contents are incorporated by reference herewith. The preferred steroidals are prednisone, prednisolone etc.

Additionally, thalidomide can be combined with or administered with and with pentoxyphylline. The amount of thalidomide is typically from about 100 mg to about 200 mg and the amount of pentoxyphylline are in the range of about 50 mg to 300 mg.

The invention also includes combinations of effective amounts of thalidomide in combination with effective amounts of levodopa and/or carbidopa. For example, a suitable formulation is 100 mg of thalidomide in combination with 100 mg of levodopa.

Thalidomide may also be combined with agents that reduce or prevent development of neurofibrillary tangles in brain tissue.

Additionally, thalidomide may be combined with agents that prevent or reduce production of TAU protein, a component of neurofibrillary tangles.

Of course, the amounts of each compound selected will depend on the weight of the mammal and the disease state. One skilled in the art can adjust the dosage forms to achieve the desired therapeutic levels.

The compound of the present invention can be prepared and administered in a wide variety of oral and parenteral dosage forms. It will be obvious to those skilled in the art that the following dosage forms may comprise as the active component, either thalidomide alone or in combination with other compounds.

Preferably the compounds of the present invention are administered orally or rectally. For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include suspensions, and emulsions, for example, water or water propylene glycol solutions. For parenteral injection, liquid preparations can be formulated in solution in aqueous polyethylene glycol solution.

Aqueous suspensions suitable for oral use can be made by dispersing the finely divided active component in water with viscous material, such as natural or synthetic gums, resins, methycellulose, sodium carboxymethylcellulose, and other well-known suspending agents. Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

It is also possible to administer thalidomide in a time-release formulation. A wide variety of methods are now available in the art for preparing time-release or long-acting compositions. Indeed, Sinamet® is available and used in a CR formulation. Any of these time-release or long-acting formulations are suitable in the practice of the present invention as long as it does not adversely affect the effectiveness of the thalidomide in the treatment of dopaminergic deficit states or other neurological states. Advantages of time-release formulations include a lower concentration of peak serum absorption which substantially reduces the adverse side effects and toxicity of the compound administered. In addition, a reduced frequency of administration results, which substantially improves patient compliance. A frequency of administration of every 12 or 24 hours would be preferred. In addition, more constant serum concentration of thalidomide would result thereby allowing a more consistent relief of symptoms.

The following examples, not to be construed as limiting, illustrate formulations which can be made according to the invention.

### EXAMPLE 1

100 mg of thalidomide are triturated and q.s. with lactose to selected capsules size.

### EXAMPLE 2

100 mg of thalidomide are mixed with 375 mg of naproxen. The active ingredients are triturated and q.s. with lactose to selected capsule size.

### EXAMPLE 3

100 mg of thalidomide are mixed with 100 mg of levodopa. The active ingredients are triturated and q.s. with lactose to selected capsule size.

The following Examples further illustrate the usefulness of the invention.

### EXAMPLE 4

Study Protocol and General Procedures

Ten-volunteers of both sexes aged 60-75 years having Hoehn & Yahr>2.5 are admitted to a single blind study, with five serving as controls. Patients underwent the following procedures in a random way:

1. Baseline: physical examination to include self reporting, blood draws, neurological assessment using UPDRS Section 3 (Motor exam), EMG (electromycardiogram) exam, skin biopsies and lumbar punctures for CSF TNF-alpha and related cytokine analysis to profile neuron cell characterization

2. The same after placebo.

3. The same after thalidomide.

Dosage

Thalidomide 200 mg once daily prior to bedtime. Dosage to be cut in half if neurotoxicity side effects, such as pheripheral neuropathy, are too difficult for patient to handle. For toxicity levels above the National Cancer Institute's Common Toxicity Criteria (CTC) Level 1, physicians are instructed to allow the patient to return to baseline and then recommence treatment with the does reduced by 50%. Minimum dosage for participation in study is 50 mg. Saline: physiological concentration.

Movement Test

The aim of this test is to study any reduction in PD movement disorders correlated with known levels of cytokines in the CSF, for patients in “off-state.” EMG Monitoring EEG monitoring starts one week after administration.

Duration of the Session: Each session lasts about one hour.

Composite Assessment

The Unified Parkinson's Disease Rating Scale (UPDRS) is a complex rating tool with numerical grades assigned throughout the longitudinal course of Parkinson's disease. The assessement tool has three sections with a possibility of 199 points: 1. Mentation, Behavior, Mood (intellectual impair, though disorder, depression, motivation/initiative); 2. Activities of Daily Living (speech, salivation, swallowing, handwriting, cutting food/handling utensils, dressing, hygiene, turning in bed/adjusting bed clothes, falling/unrelated to freezing, freezing when walking, walking, tremor, sensory complaints related to Parkinsonism; 3. Motor Exam (speech, facial expression, tremor at rest, face, right upper extremity (RUE), LUE, RLE, LLE, action or postural tremo (−RUE, −LUE), rigidity (neck, RUE, LUE, RLE, LLE), finger taps (Right, Left), hand movements (Right, Left), Leg agility (Right, Left), arising from chair, posture, gait, postural stability, body bradykinesia/hypokinesia. Rating is assigned as 100% for completely independent; 90%, completely independent but slow; 80%, independent in most chores, but slow; 70% not completely independent, very slow; 60% some dependency, slow and with effort; 50% more dependent, difficulty with everything; 40% very dependent, can assist with all chores but not alone; 30% with effort, chores now and then, much help needed; 20% nothing alone; 10% totally dependent, helpless; 0% vegetative function not working, bedridden. Although the UPDRS is the standard assessment tool, level of progression is still referred to in terms of position of 1 to 5 referring to the five stages of the Hoehn and Yahr assessment rating: Stage 1. Signs and symptoms on one side only, symptoms mild and inconvenient but not disabling, usually presents with tremor of one limb, friends have noticed changes in posture, locomotion and facial expression; Stage 2. Symptoms are bilateral, minimal disability, posture and gait are affected; Stage 3. Significant slowing of body movements, early impairment of equilibrium on walking or standing, generalize dysfunction that is moderately severe; Stage 4. Severe symptoms, can still walk to a limited extent, rigidity and bradykinesia, no longer able to live alone, tremor may be less than earlier stages; Stage 5. Cachectic stage, invalidism complete, cannot stand or walk, requires constant nursing care.

### EXAMPLE 5

Additional Study Protocol and General Procedures

The study will investigate the effect of thalidomide alone or in combination on patients with PD or other neurological diseases. The primary outcome measure used to evaluate whether thalidomide reduces TNF-α levels will be the TNF-α before and after one month of thalidomide administration. Secondary outcome measures will include correlation of CSF TNF-α with disease severity as assessed by analysis of cytokine assays and by the UPDRS motor score comparisons of adverse events in patients on thalidomide vs. placebo.

Ten (10) volunteers of both sexes aged 60-75 having Hoehn & Yahr stage 1-III are admitted to a single blind study, with five serving as controls. Patients underwent the following procedures in a random way:

1. Baseline. Physical examination to include self-reporting, blood draws, neurological assessment using UPDRS Section 3 (Motor exam),) ECG (electrocardiogram), EMG (electromyography) exams, skin biopsies and lumbar punctures for CSF TNF-alpha and related cytokine analysis to profile neuron characterization.
2. The same after placebo.
3. The same after thalidomide or after thalidomide in combination.

The UPDRS will be performed at each visit in the “off” state. This will be done in the morning before the first dose of anti-Parkinson's medication. Patients will be evaluated with general and neurological examinations, laboratory testing, ECG, and the UPDRS according to the schedule of Table 1.

TABLE 1
Schedule of assessments

| | Screening | Day 1 | Day 14 | Day 30 |
|---|---|---|---|---|
| Informed Consent | X | | | |
| Medical History | X | | | |
| Weight | X | X | | X |
| Vitals | X | X | | X |
| Exam | X | X | | X |
| Safety Labs | X | | | X |
| Urine Pregnancy | X | | | X |
| ECG | X | | | X |
| EMG | X | | | X |
| UPDRS | X | X | | X |
| Hoehn/Yahr | X | X | | X |
| Adverse events | | | | X |
| Concomitant medications | X | X | | X |
| Pill counts for compliance | | | | X |
| NCV | X | | | |
| Safety Call | | | X | |

Routine blood work including routine CBC, complete metabolic profile, coagulation profile and U/A will be done at screening visit and at 30 days.

EMG and nerve conduction studies will be performed to screen for peripheral neuropathy. Using a basic protocol under temperature-controlled conditions, one sural sensory response, a tibial motor response with F-wave and the tibial or peroneal H-reflex will be done.

Cytokine assays will be conducted based on protocols for each assay that include the sources of the antibodies, standards, and the streptavidince-biotin conjugate, the working dilution or each reagent, the standard curve range, the concentration of the internal control and the assay detection limit. CSF samples will be obtained by diagnostic lumbar puncture from patients receiving thalidomide alone or in combination therapy. CSF will be collected into sterile polystyrene tubes, sealed with screwcaps. The CSF samples will be centriguged to remove cellular debris at 10,000×g fro 5 min, the supernutant will be immediately frozen at −80° C. and stored until analysis. Repeated thawing and freezing will be avoided. TNF-α and IL-β concentrations in CSF will be analyzed by enzymen-linked immunosorbent assay (ELISA). The standard is diluted and all samples and controls are plated and read. Curve fitting is selected among linear, quandratic and 4-point based on the best regression coefficient(s).

The impact of thalidomide treatment on change from Baseline CSF TNF-α will be statistically assessed using a paired t-test. All values will be expressed as mean±standard error of the mean. Statistical analyses tests will include Mann-Whitney U test, chi-square test, or one-way analysis of variance in combination with the Tukey-Kramer multiple comparisons test. Relations between variables will be assessed by Pearson's correlation coefficient. Differences will be considered statistically significant at P<0.05.

Inclusion/Exclusion Criteria

Persons with Parkinson's or other neurological disease who have provided informed consent and are classified as Hoehn & Yahr stage I-III are included in the study.

The following persons are excluded from the study: patients with significant cardiovascular disease with a history of unstable angina, recent (<3 months) myocardial infarction, congestive heart failure or hemodynamically significant valvular disease; patients with neuropathy (based on clinical examination and baseline nerve conduction studies); women who are pregnant or breast-feeding; patients with diabetes mellitus; patients with significant dementia as defined by a MMSE<24 will be excluded; subjects with any concurrent illness that would make the use of thalidomide potentially hazardous will also be excluded.

The following restrictions are imposed on female subjects of child-bearing potential. Such subjects must have a negative urine pregnancy test at screening and must be using adequate birth control methods (double-barrier protection against conception). They must be practicing a clinically accepted method of contraception (such as an intrauterine device or diaphragm in addition to spermicidal foam and condom on the male partner, or injectable contraception, or implantable contraception, during the entire study and for at least 1 month before randomization and one month following completion of the study.

Dosage, Safety and Tolerability Considerations

Presently, there are no studies of thalidomide toxicity in patients with Parkinson's disease or other neurological diseases. As thalidomide has been used, the toxicity of thalidomide has been thoroughly evaluated for dosage, duration of dosage and interaction with concomitant drugs.

The major toxicities of thalidomide include the teratogenetic complications, neurological complications including peripheral neuropathy, somnolence, tremors, ataxia and hearing loss, gastrointestinal complications including constipation, elevated liver enzymes, vomiting and dyspepsia, dermatological complication of skin rash, thomboembolic complications of DVT and pulmonary embolism, hematological complications of neutropenia as well as sinus bradycardia, peripheral edema and orthostatic hypotension.

At each visit, a detailed medical history will be taken concerning each of these potential adverse events. Medical and neurological examinations will be done focusing on these symptoms. Side-effects are managed through dosing and duration of dosage adapted to individual patience tolerance, with 200 mg or lower considered generally non-toxic.

Thalidomide 200 mg alone or in combination once daily prior to bedtime. Dosage is to be cut in half if neurotoxcity side effects, such as pheripheral neuropathy, are too difficult for patient to handle. For toxicity levels above the National Cancer Institute's Common Toxicity Criteria (CTC) Level I, physicians are instructed to allow the patient to return to baseline and then recommence treatment with the doses reduced by 50%. Minimum dosage for participation in study is 50 mg.

Movement Test

The aim of this test is to study any reduction in PD movement disorders correlated with known levels of cytokines in the CSF, for patients in “off-state.” EMG Monitoring and ECG monitoring are conducted at screening and then on the day of administration and one month after administration, with a safety check at day 14.

From the foregoing description, one skilled in the art can easily ascertain the essential characteristics of this invention, and without departing from the spirit and scope thereof, can make various changes modifications of the invention to adapt it to various usages and conditions.

What is claimed is:

1. A method of treating the symptoms of neurological decline in a mammal, which comprises administering to a mammal affected with said neurological decline a therapeutically effective amount of thalidomide.

2. A method of treating a mammal suffering from neurological impairment associated primarily with in the population between the ages of late fifties and early sixties comprising administering to said mammal suffering from neurological impairment a therapeutically effective amount of thalidomide.

3. A method of treating the symptoms of neurological decline in a mammal which comprises administering to a mammal affected with neurological decline a therapeutically effective amount of a mixture of thalidomide with a compound selected from the group consisting of levodopa, carbidopa, non-steroidal anti-inflammatory carboxylic acids (NSAIDs), steroidal anti-inflammatory agents (SAIDs), pentoxyphylline, and a pharmaceutically acceptable inert carrier.

4. The method of claim 3 wherein said NSAID is an aryl propionic acid.

5. The method of claim 3 wherein said NSAID is an aryl acetic acid.

6. The method of claim 4 wherein said aryl propionic acid is ibuprofen.

7. The method of claim 4 wherein said aryl propionic acid is naproxen.

8. The method of claim 4 wherein said aryl propionic acid is ketoprofen.

9. The method of claim 5 wherein said aryl acetic acid is indomethacin.

10. The method of claim 3 wherein said mixture comprises thalidomide and a pharmaceutical inert carrier.

11. The method of claim 3 wherein said steroidal anti-inflammatory is prednisone.

12. The method of claim 3 wherein said steroidal anti-inflammatory is prednisolone.

13. A method for treating a central nervous system or peripheral nervous system dopaminergic deficit state or other neurological deficit state in a mammalian organism in need of such treatment, said method comprising administering to said mammal an amount of thalidomide effective in the treatment of a dopaminergic deficit state or other neurological deficit state and for a time sufficient to achieve a suitable blood level to treat said dopaminergic deficit state or other neurological deficit state.

14. The method of claim 13, comprising administering from about 50 mg to about 200 mg gram of said thalidomide per 24 hours.

15. The method of claim 14 further including a pharmaceutically acceptable inert carrier therefor.

16. The method of claim 14, comprising administering from about 50 mg to about 200 mg of said thalidomide per 24 hours.

17. The method of claim 13 further including a compound selected from the group consisting of non-steroidal anti-inflammatory carboxylic acids, levodopa, carbidopa, steroidal anti-inflammatory agents and pentoxyphilline.

18. The method of claim 17 wherein said non-steroidal anti-inflammatory carboxylic acid is ibuprofen.

19. The method of claim 17 wherein said nonosteroidal anti-inflammatory carboxylic acid is naproxen.

20. The method of claim 17 wherein said non-steroidal anti-inflammatory carboxylic acid is aspirin.

21. The method of claim 17 wherein said non-steroidal anti-inflammatory carboxylic acid is ketoprofen.

22. A method for treating Parkinson's disease in a mammal, said method comprising administering to said mammal about 50 mg to about 200 mg per 24 hours of thalidomide and a pharmaceutically acceptable inert carrier therefor.

23. A pharmaceutical composition of matter for treating central nervous system or peripheral nervous system dopaminergic deficit states or other neurological deficit states in a mammalian organism in need of such treatment, said composition comprising: (a) an effective unit dosage amount of thalidomide; (b) an additional therapeutic agent in effective amounts selected from the group consisting of prednisone, prednisolone and non-steroidal anti-inflammatory carboxylic acids, carbidopa, levodopa, and a pharmaceutical acceptable inert carrier.

24. The pharmaceutical composition of claim 23 wherein said non steroidal anti-inflammatory carboxylic acid (NSAID) is selected from the group consisting of the propionic acids, the acetic acids, the fenamic acids and the biphenyl carboxylic acids.

25. The pharmaceutical composition of claim 24 wherein said NSAID is an aryl propionic acid.

26. The pharmaceutical composition of claim 25 wherein said NSAID is ibuprofen.

27. The pharmaceutical composition of claim 25 wherein said NSAID is selected from the group consisting of indoprofen ketoprofen, naproxen, benoxaprogen, flurbiprofen, fenoprofen, fenbufen, pirprogen, carprofen, oxaprozin, pranoprofen, miroprofen, tioxaprofen, suprofen, alminoprofen, tiaprofen, fluprofen, bucloxic acid, sulindac, tolmetin, zomepirac, diclofenac, fenclofenac, alclofenac, ibufenac, isoxepac, furofenac, tiopinac, zidometacin, acematacin, fentiazac, clidanac, oxpinac, fenflozic acid, mefanamic acid, meclofenamic acid, flufenamic acid, niflumic acid, and tolfenamic acid.
